(19) United States
(12) Patent Application Publication
(10) Pub. No.: US 2007/0162119 A1
(43) Pub. Date: Jul. 12, 2007

(54) TYMPANIC MEMBRANE PROSTHESIS AND A TOOL FOR MANUFACTURING THE PROSTHESIS

(75) Inventor: Alan J. Johnson, Martinez, GA (US)

Correspondence Address: GARDNER GROFF SANTOS & GREENWALD, P.C., 2018 POWERS FERRY ROAD, SUITE 800, ATLANTA, GA, 30339, US

(73) Assignee: Medical College of Georgia Research Institute, Inc., Augusta, GA (US)

(21) Appl. No.: 11/620,811

(22) Filed: Jan. 8, 2007

Related U.S. Application Data
(60) Provisional application No. 60/756,863, filed on Jan. 7, 2006.

(51) Int. Cl. A61F 2/18

(52) U.S. Cl. 623/10; 623/901

(57) ABSTRACT

A prefabricated tympanic membrane prosthesis includes a piece of collagen sheet processed with a diluted solution of formalin. The piece of collagen sheet is molded on a forming surface of a tool having a shape resembling a natural tympanic membrane of a human subject. The piece of collagen sheet is processed with a diluted solution of formalin so that the resulting tympanic membrane prosthesis retains the shape of the forming surface. The tympanic membrane prosthesis is semi-rigid, yet flexible, and is packaged in a substantially rigid container to protect it shape during shipment and storage.

### CROSS REFERENCE TO RELATED APPLICATION

This application claims the benefit of U.S. Provisional Application Ser. No. 60/756,863, filed Jan. 7, 2006, which is hereby incorporated by reference in its entirety for all purposes.

### TECHNICAL FIELD

The present invention relates generally to a prosthetic device, and particularly to a tympanic membrane prosthesis and a tool for manufacturing the prosthesis.

### BACKGROUND OF THE INVENTION

Tympanic membrane (referred to herein as “TM”) perforation, or a perforated eardrum, is a common problem encountered in an otolaryngology practice. The treatment of these TM perforations continues to evolve with newer techniques and materials. Though fascial grafts are most commonly used today, numerous other graft materials are used, including autologous (perichondrium, fat, vein, periosteum and dura), cadaveric (fascia, dura and skin), xenograft (bovine collagen, porcine pericardium and intestinal basement membrane), and non-organic sources (paper).

Total or near total perforations represent a particularly challenging treatment situation. Lateralization and blunting are common, leading to poor hearing results. In the 1970's, a new technique was developed to address these difficulties. Formalin solution (5% formaldehyde) was used to “mold” autologous temporalis fascia (i.e., the patient's own connective tissue) in the shape of the TM using a metallic mold of the medial external auditory canal and the TM surface. The result is a semi-rigid, shaped graft that prevents blunting of the anterior sulcus. The technique has some negatives, including the presence of formalin (a dangerous chemical) near the operative site, the need for an electric heat source for drying, and an addition of 30-40 minutes to the operative time. These factors have contributed to somewhat limited acceptance of this technique.

Recently, there has been an increasing acceptance of allograft collagen in TM repair as a safe and effective alternative to fascia. Several authors have reported success rates using allograft collagen at least equal to fascia in both human and animal models. However, the use of allograft collagen alone does not provide a predefined shape for use as a tympanic membrane; rather, the practitioner must either use a flat piece of allograft collagen or shape the allograft collagen.

Thus it can be seen that needs exist for a prefabricated, “off-the-shelf” tympanic membrane prosthesis that can be implanted into a human or animal subject. It is to such provision that the present invention is primarily directed.

### SUMMARY OF THE INVENTION

Advantageously, the present invention provides a prefabricated tympanic membrane prosthesis that can be implanted in a human or animal subject who has a total or near total perforation of the tympanic membrane. The prefabrication of the tympanic membrane prosthesis occurs outside of the operating room, minimizes the amount of time in the operating room, and eliminates the presence of certain chemical hazards in the operating room, as compared to conventional techniques of creating a tympanic membrane from the subject's own skin.

In an example form, the present invention includes a tympanic membrane prosthesis having a piece of collagen sheet treated with an agent that causes the tympanic membrane prosthesis to retain a predetermined shape resembling that of a natural human tympanic membrane. Preferably, the agent is a diluted solution of formalin, such as a solution of about 4% to about 5% formalin. The tympanic membrane prosthesis can be stored in a substantially rigid container to maintain its sterility.

In another form, the present invention includes a tool for forming a tympanic membrane prosthesis. The tool has a forming surface having a shape resembling a tympanic membrane of a human subject. The forming surface further includes a plurality of perforations therein. Preferably, at least the forming surface of the tool is constructed of a nonreactive material, such as plastic, stainless steel, or titanium.

In yet another form, the present invention includes a method of fabricating a tympanic membrane prosthesis. The method includes the steps of placing a piece of collagen sheet on a mold, wherein the mold has a shape resembling that of a natural tympanic membrane of a human subject; processing the piece of collagen sheet with a buffered solution of formaldehyde so as to create a synthetic tympanic membrane prosthesis; removing the synthetic tympanic membrane prosthesis from the mold; and packaging the synthetic tympanic membrane prosthesis in a sterile container. The method can further include the steps of rehydrating the piece of collagen sheet prior to placing it on the mold; drying the piece of collagen sheet after it is placed on the mold; and trimming excess processed collagen sheet from the synthetic tympanic membrane prosthesis so as to suit a particular human subject.

These and other aspects, features and advantages of the invention will be understood with reference to the drawing figures and detailed description herein, and will be realized by means of the various elements and combinations particularly pointed out in the appended claims. It is to be understood that both the foregoing general description and the following brief description of the drawings and detailed description of the invention are exemplary and explanatory of preferred embodiments of the invention, and are not restrictive of the invention, as claimed.

### BRIEF DESCRIPTION OF THE DRAWINGS

FIG. 1 is a perspective view of a tympanic membrane prosthesis according to an example embodiment of the present invention.

FIG. 2 is a perspective view of a tool for creating the tympanic membrane prosthesis of FIG. 1, according to an example embodiment of the invention.

FIG. 3 is a perspective view of the tympanic membrane prosthesis of FIG. 1 being molded on the tool of FIG. 2.

FIG. 4 is a perspective view of a container for securely housing the tympanic membrane prosthesis of FIG. 1 therein.

FIG. 5 is a sectional view of the tympanic membrane prosthesis of FIG. 1 implanted in an ossicular system of a human subject.

### DETAILED DESCRIPTION OF EXAMPLE EMBODIMENTS

The present invention may be understood more readily by reference to the following detailed description of the invention taken in connection with the accompanying drawing figures, which form a part of this disclosure. It is to be understood that this invention is not limited to the specific devices, methods, conditions or parameters described and/or shown herein, and that the terminology used herein is for the purpose of describing particular embodiments by way of example only and is not intended to be limiting of the claimed invention. Also, as used in the specification including the appended claims, the singular forms “a,” “an,” and “the” include the plural, and reference to a particular numerical value includes at least that particular value, unless the context clearly dictates otherwise. Ranges may be expressed herein as from “about” or “approximately” one particular value and/or to “about” or “approximately” another particular value. When such a range is expressed, another embodiment includes from the one particular value and/or to the other particular value. Similarly, when values are expressed as approximations, by use of the antecedent “about,” it will be understood that the particular value forms another embodiment.

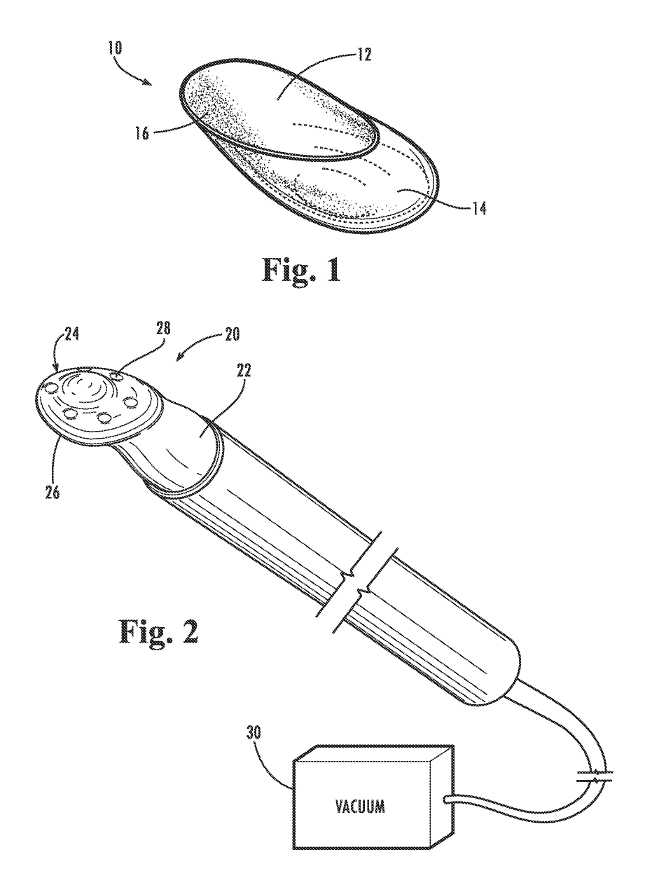

FIG. 1 depicts a perspective view of a tympanic membrane prosthesis 10 according to an example embodiment of the present invention. In the depicted embodiment, the tympanic membrane prosthesis 10 is formed of a generally rounded rectangular piece of collagen sheet 12 (which shape can generally resemble a human thumbnail), such as but not limited to allograft collagen or acellular dermal matrix, having a diameter of about 10 mm and a thickness of about 0.006 inch to about 0.012 inch, although those skilled in the art will understand that such dimensions are exemplary. Preferably, the piece of collagen sheet 12 is a conventional acellular dermal product, such as ALLODERM®, as manufactured by LifeCell Corporation of Branchburg, N.J., although those skilled in the art will understand that various other collagen sheets can be used as well. Generally, conventional collagen sheet products are freeze-dried. In the present invention, the piece of collagen sheet 12 is preferably rehydrated according to the manufacturer's directions prior to molding it.

The tympanic membrane prosthesis 10 generally resembles that of a natural tympanic membrane in size and shape. The tympanic membrane prosthesis 10 has a first curved wall 14 connected a second curved wall forming a skirt 16 with a shape similar to that of the medial portion of the external ear canal. The skirt 16 serves to facilitate surgical implantation by providing a structure for interfacing with host tissue. The tympanic membrane prosthesis 10 is semi-rigid, yet flexible, and has a stiffness such that it can retain its shape.

In an example method of forming the tympanic membrane prosthesis 10, a tool 20 or mold is used, as depicted in FIG. 2. The tool 20 includes a generally-cylindrically shaped shaft 22, which can approximate the size and shape of a human external ear canal. At a distal end 24 of the shaft 22 is a head or forming surface 26 having a surface that corresponds to the shape of a natural tympanic membrane. Preferably, at least the forming surface 26 of the tool 20 is constructed of a nonreactive material, such as plastic, stainless steel, or titanium, although those skilled in the art will understand that various other nonreactive materials can be employed as well. Preferably, the shaft 22 and the forming surface 26 are hollow and define an interior chamber of the tool 20. Also preferably, the forming surface 26 of the tool 20 further includes a plurality of perforations 28 or holes extending therethrough, in communication with the interior chamber of the tool 20. In the depicted embodiment, the perforations 28 are a plurality of generally circular-shaped openings located on a first face of the forming surface 26; however, those skilled in the art will understand that perforations of various shapes and sizes can be employed as well. For example, a plurality of slits can be employed. The shaft 22 of the tool 20 can be connected to a vacuum source 30 and/or a heat source such that suction and/or heat can be applied through the perforations 28.

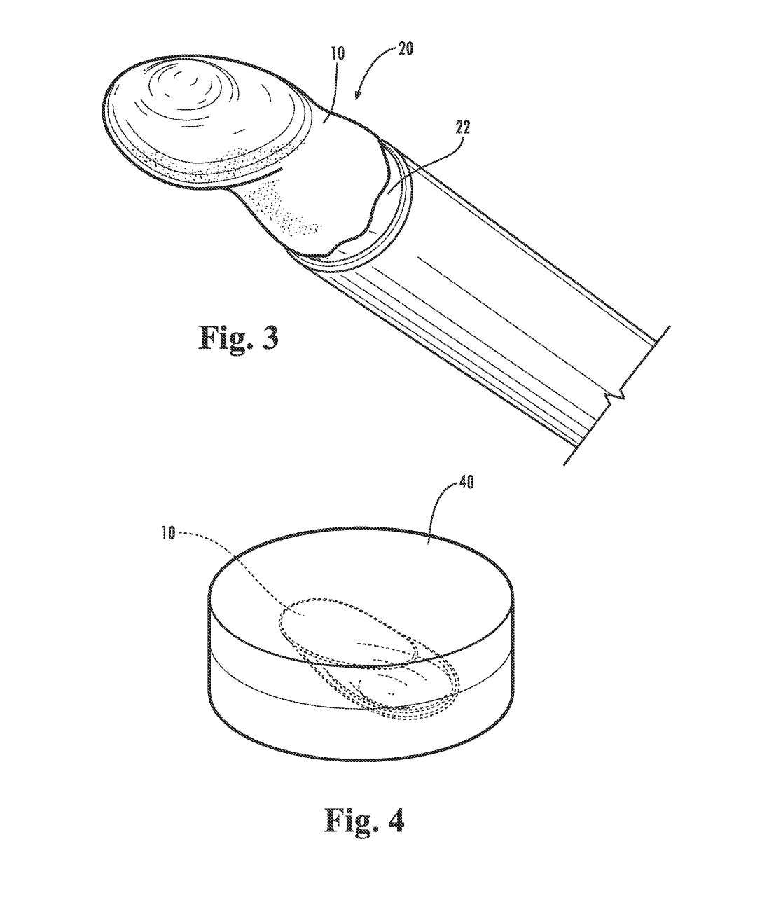

FIG. 3 depicts a perspective view of the tympanic membrane prosthesis 10 being molded on the tool 20. The rehydrated piece of collagen sheet 12 (i.e., rehydrated per the manufacturer's directions), which is wet, is placed over the forming surface 26 of the distal end 24 of the tool 20. The surface tension of the wet collagen sheet on the forming surface 26 is substantially sufficient to hold the wet material on the forming surface. Additionally, suction provided by the vacuum 30 further serves to secure the collagen sheet 12 to the forming surface 26. Optionally, a retaining sleeve can be placed around the collagen sheet 12 so as to further secure the collagen sheet to the forming surface 36. The wet piece 12 is dried by applying suction from the vacuum 30 through the perforations 28. Optionally, the vacuum 30 can include a heat source such that heat is applied through the perforations 28 to dry the collagen sheet 12. Alternatively, a separate heat source, such as a heat lamp, can be provided to dry the collagen sheet 12. In an alternative method, the wet piece of collagen sheet 12 can be press-molded between male and female die members.

The piece of collagen sheet 12 is processed with an agent, which causes the collagen sheet to retain the configuration of the forming surface 26 of the tool 20. The forming surface 26 with the piece of collagen sheet 12 thereon can be immersed in a container of or sprayed with the agent. Preferably, the agent is a diluted or buffered solution of formaldehyde, such as about 5% formalin, which results in the cross-linking of disulfide bonds in the collagen sheet material. For example, the tympanic membrane prosthesis 10 can be dipped into an about 5% formalin solution for approximately eight minutes, and then removed from the formalin. Preferably, the collagen sheet 12 remains exposed to the fixative solution until adequate fixation has occurred rendering the new shape to be that of the forming surface 26 of the tool 20. The collagen sheet 12 and tool 20 can then be submerged in a water bath for approximately 5-8 minutes. Preferably, this process of submerging the collagen sheet 12 and forming surface 26 in a fresh water bath is repeated three times until negligible amounts of residual formalin remain. The tympanic membrane prosthesis 10 is then removed from the forming surface 26 of the tool 20. Alternatively, the tympanic membrane prosthesis 10 can be removed from the forming surface 26 of the tool 20 after it is dipped in the formalin but prior to submerging it in the water bath. Preferably, the tympanic membrane prosthesis 10 is then air-dried, although those skilled in the art will understand that heat could be applied to dry the prosthesis.

Another suitable solution for treating the piece of collagen sheet 12 is about 4% formaldehyde buffered to a pH of about 5.0 to about 7.0, although those skilled in the art will understand that the percentage and pH can vary. Alternatively, a functionally similar fixative such as, but not limited to, gluteraldehyde can be used.

The tympanic membrane prosthesis 10 retains the shape of the forming surface 26 of the tool 20. The resulting tympanic membrane prosthesis 10 is a generally semi-rigid, yet flexible, prosthesis that generally resembles the size and shape of a natural human tympanic membrane. Those skilled in the art will understand that one or more steps of the process of fabricating the tympanic membrane prosthesis 10 can be automated.

FIG. 4 depicts a perspective view of a container 40 for housing the tympanic membrane prosthesis 10. Preferably, the container 40 is rigid or substantially rigid so as to protect the tympanic membrane prosthesis 10 during shipment and storage of the prosthesis. An exemplary material used to construct the container is plastic, although those skilled in the art will understand that other semi-rigid or rigid materials can be employed as well. Those skilled in the art will further understand that although the shape of the container 40 depicted in FIG. 4 is generally cylindrical, a variety of other shapes and sizes can be employed as well. Alternatively, the container 40 can be sealed in flexible packaging. Preferably, the container 40 is sealed in a manner so as to preserve the sterility of the tympanic membrane prosthesis 10. Also preferably, the tympanic membrane prosthesis 10 is stored dry.

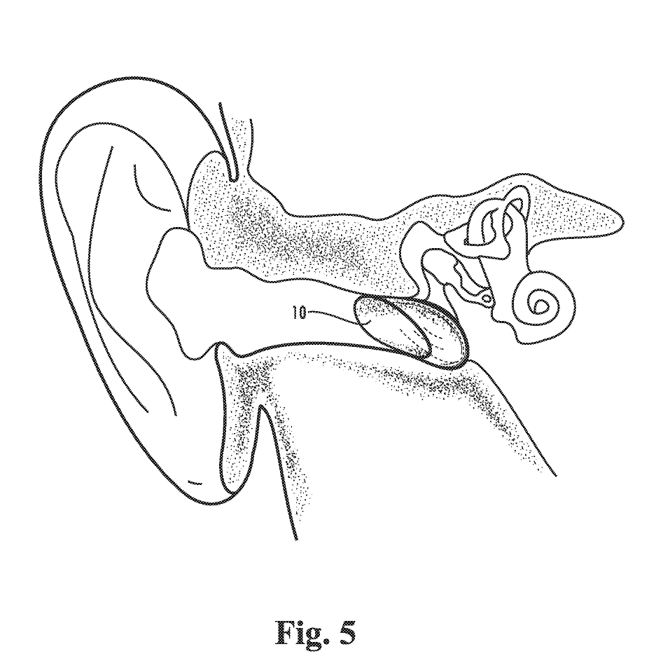

FIG. 5 depicts a sectional view of the prefabricated tympanic membrane prosthesis 10 implanted in an ossicular system of a human subject. In an example method of use, a practitioner breaks the seal of the container 40 and removes the tympanic membrane prosthesis 10. The practitioner can then optionally trim the skirt 16 to suit a particular patient such that the tympanic membrane prosthesis 10 provides a form fit. Alternatively, a series of implants of various sizes and configurations are provided, such that a practitioner can select the desired implant from the series. The practitioner can then implant the prosthesis 10 in accordance with conventional techniques.

While the invention has been described with reference to preferred and example embodiments, it will be understood by those skilled in the art that a variety of modifications, additions and deletions are within the scope of the invention, as defined by the following claims.

What is claimed is:

1. A tympanic membrane prosthesis, comprising:
a piece of collagen sheet treated with an agent that causes the collagen sheet to retain a predetermined shape.

2. The tympanic membrane prosthesis of claim 1, wherein the agent is a diluted solution of formalin.

3. The tympanic membrane prosthesis of claim 1, wherein the agent is a solution of about 4% to about 5% formalin.

4. In combination, the tympanic membrane prosthesis of claim 1 and a container, wherein the tympanic membrane prosthesis is stored in the container to maintain its sterility.

5. The tympanic membrane prosthesis of claim 4, wherein the container is substantially rigid.

6. The tympanic membrane prosthesis of claim 1, wherein the piece of collagen sheet has a diameter of about 10 mm.

7. The tympanic membrane prosthesis of claim 1, wherein the piece of collagen sheet has a thickness of about 0.006 to about 0.012 inch.

8. The tympanic membrane prosthesis of claim 1, wherein the piece of collagen sheet is formed by vacuum molding.

9. A tool for forming a tympanic membrane prosthesis, comprising:
a forming surface having a shape resembling a tympanic membrane of a human subject and comprising a plurality of perforations therein.

10. The tool of claim 9, wherein the forming surface comprises an external portion of a hollow shaft, and wherein an inner chamber of the shaft is in fluid communication through a plurality of perforations in the forming surface.

11. The tool of claim 10, wherein the shaft further comprises a coupling for connection to a vacuum source.

12. The tool of claim 9, wherein the forming surface is constructed of a nonreactive material.

13. The tool of claim 12, wherein the nonreactive material is one of plastic, stainless steel, or titanium.

14. The tool of claim 9, wherein the plurality of perforations include a plurality of circular openings.

15. A method of fabricating a tympanic membrane prosthesis, comprising:
placing a piece of collagen sheet on a mold, wherein the mold has a shape resembling that of a natural human tympanic membrane;
processing the piece of collagen sheet with a buffered solution of formaldehyde so as to create a synthetic tympanic membrane prosthesis;
removing the synthetic tympanic membrane prosthesis from the mold; and
packaging the synthetic tympanic membrane prosthesis in a sterile container.

16. The method of claim 15, wherein the sterile container is substantially rigid.

17. The method of claim 15, wherein the buffered solution of formaldehyde further comprises a buffered solution of about 4% to about 5% formaldehyde.

18. The method of claim 15, further comprising trimming excess processed collagen sheet from the synthetic tympanic membrane prosthesis so as to suit a particular human subject.

19. The method of claim 15 further comprising rehydrating the piece of collagen sheet prior to placing it on the mold.

20. The method of claim 19 further comprising drying the piece of collagen sheet after it is placed on the mold.
